(19) United States
(12) Patent Application Publication
(10) Pub. No.: US 2016/0346334 A1
(43) Pub. Date: Dec. 1, 2016

(54) EXOSOMES AS A THERAPEUTIC FOR CANCER

(71) Applicant: STC.UNM, Albuquerque, NM (US)

(72) Inventor: Kristina Trujillo, Placitas, NM (US)

(21) Appl. No.: 15/116,579

(22) PCT Filed: Feb. 5, 2015
(86) PCT No.: PCT/US2015/014623

§ 371 Date: Aug. 4, 2016

Related U.S. Application Data
(60) Provisional application No. 62/047,297, filed on Sep. 8, 2014.
(60) Provisional application No. 61/936,095, filed on Feb. 5, 2014.

(51) Int. Cl. A61K 35/55; A61K 9/00; A61K 45/06

(52) U.S. Cl. CPC A61K 35/55; A61K 45/06; A61K 9/0019

(57) ABSTRACT

In one embodiment, the invention provides substantially purified exosomes which induce apoptosis in breast cancer cells and which are derived from a cultured medium of histologically normal breast tissue cells that are obtained from tumor-adjacent normal breast tissue. Related methods of treating breast cancer and pharmaceutical formulations are also provided.

### CROSS-REFERENCE TO RELATED APPLICATIONS AND GOVERNMENT SUPPORT

This application claims priority from both U.S. Provisional Patent Application No. 61/936,095, entitled “Exosomes from Field Cancerized Tissue”, filed Feb. 5, 2014 and U.S. Provisional Patent Application No. 62/047,297, entitled “Exosomes as a Therapeutic for Cancer”, filed Sep. 8, 2014. The complete contents of each of these provisional patent applications are hereby incorporated by reference in their entirety.

This invention was made with government support under grant no. 5R21CA17107302 awarded by the National Institute of Health/National Cancer Institute. The government has certain rights in the invention.

### FIELD OF THE INVENTION

The present invention is directed to compositions which comprise tumor-derived exosomes and methods of using these compositions in the treatment of cancer. In one embodiment, the invention provides substantially purified exosomes which induce apoptosis in breast cancer cells and which are derived from a cultured medium of histologically normal breast tissue cells that are obtained from tumor-adjacent normal breast tissue, in preferred embodiments, from a human patient with breast cancer. In still other embodiments the substantially purified exosomes are obtained from the same human patient with breast cancer who is to be treated (“autologous exosomes”). Related methods of treating breast cancer and pharmaceutical formulations are also provided.

### BACKGROUND OF THE INVENTION

An average person will develop many cancerous growths over the course of a lifetime. Currently used chemotherapeutic agents result in considerable side effects due to their lack of tumor specificity. Clinically available targeted therapies are designed to target single molecules, which could result in selection of cell populations lacking the target, and eventual drug resistance. In contrast, a vast majority of these cancers will be identified and eliminated by the human body's intrinsic cancer-protection mechanisms.

The majority of breast cancers occur in postmenopausal women, with 75% of these tumors being estrogen dependent as defined as estrogen receptor (ER) positive. Tamoxifen, an anti-estrogen, has been the mainstay of treatment for hormone-dependent breast cancers. However, recent clinical trials have shown that inhibitors of aromatase, which catalyze the rate-limiting step of estrogen biosynthesis, may be more effective than tamoxifen in treating hormone-dependent breast cancers in postmenopausal women. Unfortunately, resistance to both these endocrine therapies is inevitable in metastatic breast cancer.

Exosomes are small (30 nm-250 nm) vesicles secreted by most cell types. Recent studies have demonstrated an important role for tumor-derived and fibroblast-derived exosomes in promoting tumor progression 1, 2. As a result, there have been efforts to identify drugs that inhibit exosome production (Abdel-Mageen, 1UH2TR000928 NCATS). However, there have also been studies demonstrating that tumor cells can be manipulated to produce exosomes with anti-tumor activity 3, 4.

While most efforts to harness the human body's cancer-defense mechanisms have focused on the immune system, the present invention provides for the use of a composition that comprises exosomes, method of producing and method of using exosomes for the treatment of neoplastic cancer and tumors. There remains a particularly compelling need for breast cancer therapies such as those described herein, especially insofar as they can be used in treating patients who have acquired anti-estrogen resistance and/or an intrinsic resistance to anti-estrogen and anti-HER2 therapies.

### SUMMARY OF THE INVENTION

There is a growing body of evidence demonstrating that tissue adjacent to breast tumors, although histologically normal, possesses many of the molecular abnormalities found in patient matched tumor tissues. We have discovered that the epithelial cells demonstrate properties of tumor cells such as genomic instability (1A), expression of telomerase (2A-3A), and epithelial to mesenchymal transition (4A). We have found that the fibroblasts exhibit properties of Carcinoma Associated Fibroblasts (CAFs) such as wound healing gene expression, secretion of dense extracellular matrix, and the ability to contract (4A) which are known to promote tumorigenesis in adjacent cells. These alterations decrease as a function of distance from the tumor. We term tumor adjacent tissue with these characteristics Field Cancerized Tissue (FCT). We deduced that phenotypically normal cells outside the tumor margins are primed to promote (fibroblasts) or undergo neoplastic transformation (epithelia) by performing the first functional analysis of tumorigenic properties of epithelia and fibroblasts from FCT in vitro.

We have identified the innate production of exosomes with anti-tumor activity from a population of fibroblasts in the tumor microenvironment and we have demonstrated the effect of exsosomes derived from human primary cancer fibroblasts, as well as fibroblasts derived from Tumor Adjacent Histologically Normal tissue 1 cm, 3 cm and 5 cm from the tumor (TAHN-1, TAHN-3, TAHN-5, respectively) on breast epithelial cells. We have discovered that conditioned media (CM) from tumor and TAHN-1 fibroblasts have tumor-promoting properties, causing an increase in proliferation and migration in non-malignant MCF10a and malignant MCF7 and MDA-MB231 cells. These effects are lost upon removal of exosomes. Conversely, we have demonstrated that CM from TAHN-5 fibroblasts selectively inhibits proliferation and migration in malignant cells, but not in MCF10a non-malignant cells. This effect is also lost upon removal of exosomes from the CM.

Accordingly, in one embodiment, the present invention provides for a method for treating a neoplastic cancer in an animal, comprising administering to the animal (human or non-human) in need thereof an effective amount of an exosome from a fibroblast obtained from histologically normal tissue within a neoplastic cancer affected organ of the animal. The step of administering may include direct application to or direct injection into a tumor, or intravenous administration or delivery via the lymphatic circulation system but not limited thereto. The fibroblast may be derived from tissue at a distance of greater than about 2 cm (often about 3 cm to about 10 cm, more often about 3 cm to about 6 cm and most often about 5 cm) from a tumor in the cancer affected organ in the animal. Alternatively, the fibroblast is derived from tissue at a distance of between about 3-6 cm from a tumor in the cancer affected organ in the animal. For example, the cancer affected organ in the animal is a mammary organ such as the breast.

In another example the histologically normal tissue of the cancer affected organ is located outside of a field cancerized tissue. The fibroblast may further be treated with tumor secretions or cancer cell conditioned media or drugs to induce exosomes that are cytotoxic to the tumor cells. In another embodiment the exosome may be isolated or may be in a fluid that bathes the fibroblast. In another example the fibroblast is from a TAHN-5 cell line.

Another embodiment provides for a method of producing whole exosomes with anti-tumor properties comprising growing in culture fibroblasts derived from histologically normal tissue of a cancer affected organ from an animal. Secreted substances from the cell culture are collected. The exosomes are separated from other secreted substances and the separated exosomes are collected. In one example the collection step includes centrifuging the secreted substance to obtain the exosomes. The fibroblast may be derived from tissue at a distance of greater than about 2 cm from a tumor in the cancer affected organ in the animal. Alternatively, the fibroblast is derived from tissue at a distance of between about 3-10 cm, often about 3-6 cm and most often about 5 cm from a tumor in the cancer affected organ in the animal. For example, the cancer affected organ in the animal is a mammary organ for example the breast but not limited thereto as other cancer affected organs could benefit such as the colon, prostate, pancreas, liver, and lung. In another example the histologically normal tissue of the cancer affected organ is located outside of a field cancerized tissue. The fibroblast may further be treated with tumor secretions or cancer cell conditioned media. In another embodiment the exosome may be isolated or may be in a fluid that bathes the fibroblast. In another example the fibroblast is from a TAHN-5 cell line. For example the TAHN-5 cell line is immortalized.

Another embodiment provides for a method of producing exosomes comprising culturing fibroblasts harvested from an organ of an animal that does not have cancer or is histologically normal. The fibroblasts are treated with tumor secretions or cancer cell conditioned media. The exosomes from the treated fibroblast culture media are harvested. For example, the fibroblasts are an immortalized cell line.

Another embodiment of the present invention provides for an exosome isolated from a TAHN-5 cell line. For example the TAHN-5 cell line is immortalized. Alternatively an exosome as produced according to one of the embodiments described for making the same.

Another embodiment of the present invention provides for a pharmaceutical composition comprising an exosome according to any one of the embodiments described and a pharmaceutically-acceptable vehicle, carrier, or excipient.

In still another aspect, the present invention provides for the use of exosomes as a direct therapy for neoplastic cancer treatment. Another aspect of the present invention provides for the use of exosomes to inhibit migration and/or proliferation of tumor cells. And a further aspect of the present invention provides exosomes derived from TAHN fibroblast populations that have cytotoxic effects on breast cancer cells. The cytotoxicity varies by altering the distance of the fibroblast from a tumor, and/or varying patient populations from which the fibroblast are derived.

In a preferred embodiment, the invention provides substantially purified exosomes which induce apoptosis in breast cancer cells and which are derived from a cultured medium of histologically normal breast tissue cells that are obtained from tumor-adjacent normal breast tissue. Preferably, the histologically normal breast tissue cells: (1) are obtained from normal breast tissue located approximately 5 cm from a breast cancer tumor (2) are obtained from breast branching epithelium (terminal duct lobular units (TDLUs)) and/or surrounding stroma, and (3) are selected from the group consisting of luminal epithelial cells, myoepithelial cells, fibroblasts, immune cells, endothelial cells and extracellular matrix cells.

In certain embodiments, the breast cancer tumor is associated with invasive ductal carcinoma, ductal carcinoma in situ (DCIS) or invasive lobular carcinoma and expresses or is associated with one or more breast tumor-associated antigens or compositions selected from the group consisting of epidermal growth factor receptor EGFR, HER/neu, CR1, M18, M39, HER2 antigen (p185HER2), polymorphic epithelial mucin (PEM) (Hilkens et al., 1992, Trends in Bio. Chem. Sci. 17:359), carcinoma embryonic antigen (CEA), prostate specific antigen (PSA) Erb B2 antigen, gross cystic disease fluid protein-15 (GCDFP-15), lactose dehydrogenase (LDH), circulating tumor DNA CA 15-3, carcinoembryonic antigen (CEA), cancer antigen 125 (CA 125), Survivin, MUC1, CD44, CD24, oestrogen receptor alpha (ERα), CA15-3, TPA, TPS, Urokinase plasminogen activator (uPA) and plasminogen activator inhibitor (PAI-1). In certain embodiments, the breast cancer tumor is a primary tumor which is triple-negative (lacking ER, PR, HER2), hormone resistant (SERM-, SERD-, or AI-resistant), kinase inhibitor resistant, or a metastatic breast cancer tumor.

Exosomes of the invention can be loaded with a small molecule, antisense oligonucleotide, siRNA, peptide, protein or antibody that inhibits the growth of, or induces apoptosis in, breast cancer cells. Useful antibodies include, but are not limited to, a humanized monoclonal antibody or a F(ab′)₂ or Fab′ fragment thereof. For example, the antibody can be selected from the group consisting of trastuzumab, Pertuzumab ado-trastuzumab and emtansine (Kadcyla®). Representative small molecules are selected from the group consisting of tamoxifen, paclitaxel and fluorouracil (5-FU).

In certain embodiments, the exosomes induce apoptosis in MCF7 and MDA-MB-231 breast cancer cells.

These and other aspects of our invention are described further in the Detailed Description of the Invention.

### BRIEF DESCRIPTION OF THE FIGURES

FIG. 1. Fibroblasts from FCT produce exosomes. Electron microscopy of exosomes from tumor and FCT tissue. Size characterization shows differences in distribution of size of exosomes.

FIG. 2. TAHN-3 and TAHN-5 CM with exosomes decreases MCF7 cell growth. MCF cells were treated with CM from tumor, TAHN-1, TAHN-3 and TAHN-5 fibroblasts with and without exosomes. Cell confluence was measured using live cell imaging. Dashed line indicates least squares mean for tumor. Differences of least squares means for the indicated comparisons are shown in the corresponding table. Data shown are compiled from 3 patient sets of tumor and matched adjacent tissues.

FIG. 3. Malignant MDA-MB231 cells were treated with conditioned media (CM) from fibroblasts derived from tumor and patient-matched TAHN-1, TAHN-3 and TAHN-5 tissues (a) with exosomes and (b) without exosomes. Cytotoxicity was measured using CeiiTox reagent and Green Fluorescence was measured in the IncuCyte Zoom instrument.

FIG. 4. Non-malignant MCF10a cells were treated with conditioned media (CM) from fibroblasts derived from tumor and patient-matched TAHN-1, TAHN-3 and TAHN-5 tissues (a) with exosomes and (b) without exosomes. Cytotoxicity was measured using CeiiTox reagent and Green Fluorescence was measured in the IncuCyte Zoom instrument.

FIG. 5. Malignant MDA-MB231 cells were treated with conditioned media (CM) from fibroblasts derived from tumor and patient-matched TAHN-1, TAHN-3 and TAHN-5 tissues (a) with exosomes and (b) without exosomes. Apoptosis was measured using Cell Player Kinetic Caspase 3/7 Assay and Green Fluorescence was measured in the IncuCyte Zoom instrument.

FIG. 6. (A) Non-tumorigenic MCF 10A cells were treated with media conditioned by fibroblasts from CAFs, TAHN-1, TAHN-3 and TAHN-5 tissues and migration was measured using a scratch assay. (B) Conditioned Media (CM) was centrifuged at 10,000 g for 45 minutes and the resulting pellet was removed. The scratch assay was performed on MCF10A cells treated with CM (+) and with the centrifuged media (−). Results shown are from 5 patients.

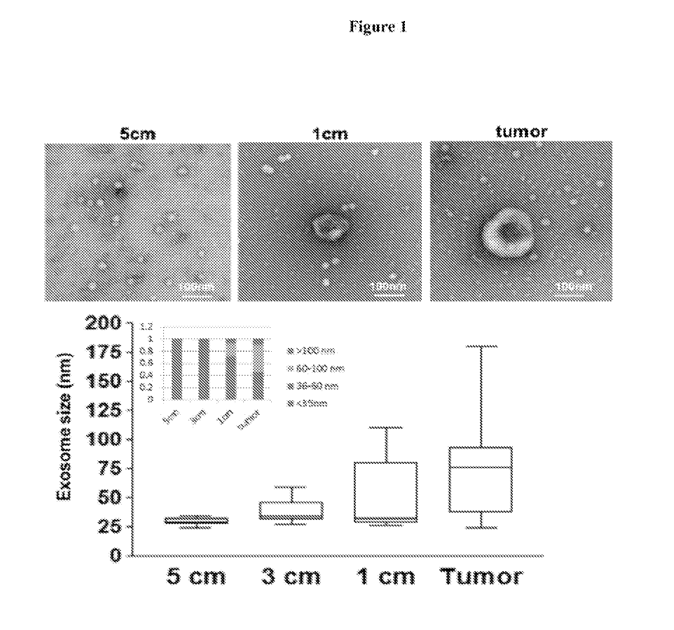

FIG. 1X. Morphology of MCF7 and MDA-MB-231 breast cancer cells treated with TAHN-5 fibroblast CM with and without exosomes.

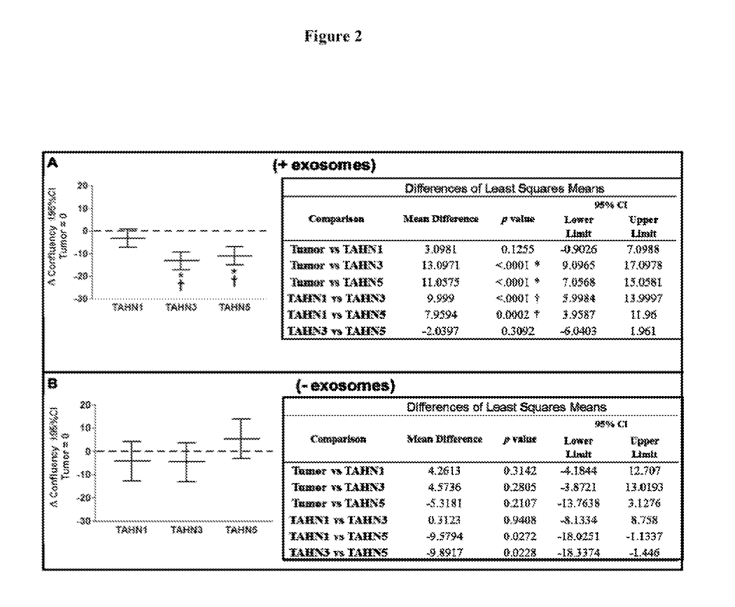

FIG. 2X. Electron microscopy of exosomes derived from TAHN-5 fibroblasts.

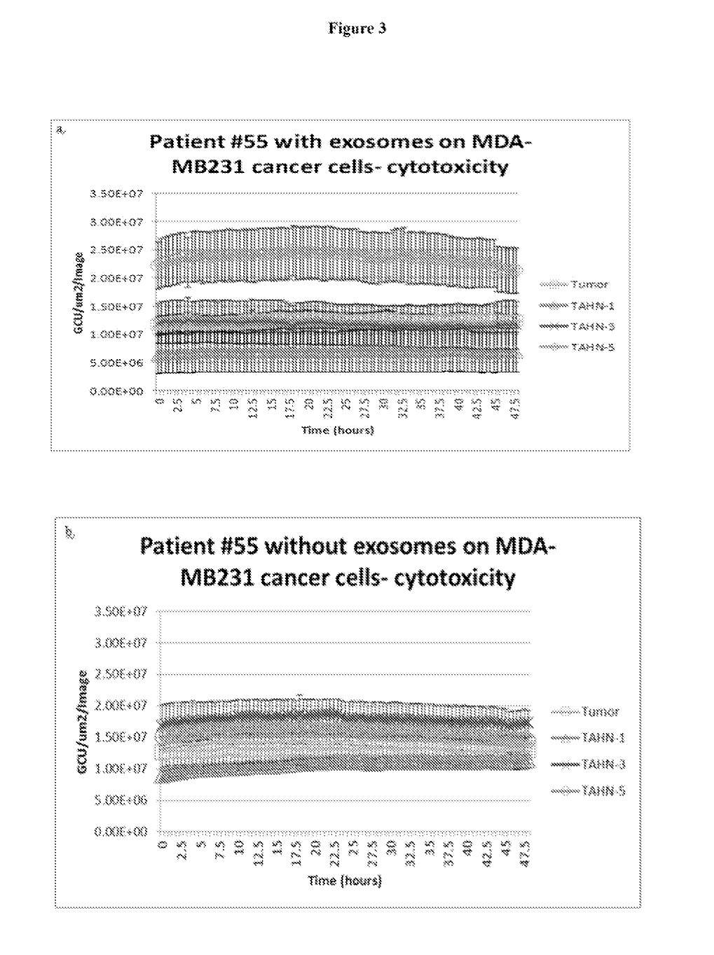

FIG. 3X. Preliminary data showing that a statistically significant higher amount of apoptosis took place in the THAN-5 fibroblast population.

### DETAILED DESCRIPTION OF THE INVENTION

In accordance with the present invention there may be employed conventional chemical synthetic methods and other biological and pharmaceutical techniques within the skill of the art. Such techniques are well-known and are otherwise explained fully in the literature.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “and” and “the” include plural references unless the context clearly dictates otherwise.

Furthermore, the following terms shall have the definitions set out below. It is understood that in the event a specific term is not defined hereinbelow, that term shall have a meaning within its typical use within context by those of ordinary skill in the art.

The term “compound”, as used herein, unless otherwise indicated, refers to any specific chemical compound disclosed herein. Within its use in context, the term generally refers to a single compound comprising a hydrophobic moiety and a linker which is capable of reacting and forming a covalent bond with a fusion protein as otherwise described herein. In certain instances the term may also refer to stereoisomers and/or optical isomers (including racemic mixtures) or enantiomerically enriched mixtures of disclosed compounds. Compounds which are disclosed are those which are stable and where a choice of substituents and claim elements is available, the substituent or claim element is chosen such that stable compounds are formed from the disclosed elements and substituents. The symbol [symbol] in a chemical structure or formula signifies that either a double or single bond may be present between the atoms to which such symbol is attached, depending upon the valence of those atoms and substituents which are on such atoms.

It should be recognized that compounds referred to herein can contain chiral carbon atoms. In other words, it may have optical isomers or diastereoisomers.

The term “patient” or “subject” is used throughout the specification within context to describe an animal, especially including a domesticated mammal and preferably a human, to whom a treatment or procedure, including a prophylactic treatment or procedure is performed. For treatment of those infections, conditions or disease states which are specific for a specific animal such as a human patient, the term patient refers to that specific animal. In most instances, the patient or subject of the present invention is a domesticated/agricultural animal or human patient of either or both genders.

The term “effective” is used herein, unless otherwise indicated, to describe an amount of a compound or composition which, in context, is used to produce or effect an intended result, whether that result relates to the treatment of a cancer in a patient or subject. The term effective subsumes all other effective amount or effective concentration terms which are otherwise described or used in the present application.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Cancers generally show partial or complete lack of structural organization and functional coordination with the normal tissue and most invade surrounding tissues, metastasize to several sites, and are likely to recur after attempted removal and to cause the death of the patient unless adequately treated. As used herein, the term cancer is used to describe all cancerous disease states applicable to treatment according to the present invention and embraces or encompasses the pathological process associated with all virtually all epithelial cancers, including carcinomas, malignant hematogenous, ascitic and solid tumors. Examples of cancers which may be treated using methods according to the present invention include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas. See, for example, The Merck Manual of Diagnosis and Therapy, 17.sup.ed. (Whitehouse Station, N.J.: Merck Research Laboratories, 1999) 973-74, 976, 986, 988, 991).

In addition to the treatment of ectopic cancers as described above, the present invention also may be used preferably to treat eutopic cancers such as choriocarcinoma, testicular choriocarcinoma, non-seminomatous germ cell testicular cancer, placental cancer (trophoblastic tumor) and embryonal cancer, among others.

“The human breast gland is composed of two main cellular compartments, the branching epithelium, commonly referred to as the terminal duct lobular units (TDLUs) and the surrounding stroma. The TDLUs consist of an inner layer of luminal epithelial cells and an outer layer of myoepithelial cells separated from the surrounding vascular rich stroma by a basement membrane. The breast stroma is composed of cellular components such as fibroblasts, immune cells and endothelial cells and the extracellular matrix (ECM) as well as entrapped growth. factors within the ECM. Breast stroma accounts for roughly 80% of the total tissue volume and exerts a dominant effect on tissue morphogenesis in both the normal and malignant breast gland.” Ingthorsson, et al., “Endothelial cells stimulate growth of normal and cancerous breast epithelial cells in 3D culture”, BMC Research Notes 2010, 3:184 (citations omitted).

As explained in U.S. Patent Application Document No. 20140350102, “the standard of care [of breast cancer patients] currently includes screening the tumor for the expression levels of the hormone receptors, estrogen receptor (ER) and progesterone receptor (PR), and the human epidermal growth factor receptor 2 (HER2) kinase. Currently, a woman diagnosed with breast cancer may be treated preliminarily with surgery, chemotherapy (optional in some cases), and radiation before targeted therapy is initiated. Hormone receptor positive breast cancers are susceptible to hormone therapies with selective estrogen receptor modulators or SERMs (e.g., tamoxifen, toremifene), aromatase inhibitors (e.g., anastrozole), or selective estrogen receptor degraders or SERDs (e.g., fulvestrant). Hormone therapies such as aromatase inhibitors (AI) block production of estrogens in the body (typically used in post-menopausal women), whereas SERMs and SERDs block the proliferative action of estrogens on the breast cancer cells. HER2 positive breast cancers are susceptible to HER2 kinase inhibitors (e.g., trastuzumab and lapatinib) and are generally used in metastatic disease. Anti-angiogenic therapy (bevacizumab) is also approved in metastatic disease. Despite these multiple tiers of targeted treatments, patients often have or develop refractory forms of breast cancer. Examples of refractory breast cancer include primary tumors which are triple-negative (lacking ER, PR, HER2), hormone resistant (SERM-, SERD-, or AI-resistant), or kinase inhibitor resistant, or metastatic breast cancer tumors. Once the targeted therapies fail or tumors metastasize, radiation and high dose chemotherapy are required to ablate the refractory breast cancer tumors. Current chemotherapies available for the treatment of refractory breast cancer include anthracyclines, taxanes, and epothilones, which are toxic, dangerous, costly, and often are ineffective, especially in the treatment of metastatic disease.

Abundant clinical evidence suggests that androgens normally inhibit breast growth. For instance, women with androgen deficits have an increased risk for developing breast cancer. Androgen signaling plays a crucial role in breast homeostasis, negating the proliferative effects of estrogen signaling in the breast. However, when androgens transform into estrogens (aromatase pathway), they increase cell proliferation and mammary carcinogenesis risk. Historically, the steroidal androgen receptor agonists testosterone, fluoxymesterone, and calusterone were used in advanced breast cancer. These agents suffered from side effects such as excessive virilization, cross-reactivity with the estrogen receptor, and aromatization to estrogens. The use of steroidal androgens in advanced breast cancer pre-dates the screening of breast cancers for hormone and kinase receptors. Recently, it was found that the AR is expressed in 50-90% of breast tumors, providing a mechanism to use androgens as targeted therapy for AR-positive breast cancers.

Selective androgen receptor modulators (SARMs) are compounds which demonstrate AR-mediated tissue selective activity. Unlike their steroidal precursors, SARMs are non-aromatizable, generally demonstrate no activity at other steroidal receptors including ER and PR, and are non-virilizing. Further, SARMs may be beneficial in refractory breast cancer patients due to their hypermyoanabolic effects that should improve their tolerance of high-dose chemotherapy.”

A breast cancer tumor treated with exosomes of the invention may express or be associated with one or more breast tumor-associated antigens or compositions selected from the group consisting of epidermal growth factor receptor EGFR, HER/neu, CR1, M18, M39, HER2 antigen (p185HER2), polymorphic epithelial mucin (PEM) (Hilkens et al., 1992, Trends in Bio. Chem. Sci. 17:359), carcinoma embryonic antigen (CEA), prostate specific antigen (PSA) Erb B2 antigen, gross cystic disease fluid protein-15 (GCDFP-15), lactose dehydrogenase (LDH), circulating tumor DNA CA 15-3, carcinoembryonic antigen (CEA), cancer antigen 125 (CA 125), Survivin, MUC1, CD44, CD24, oestrogen receptor alpha (ERα), CA15-3, TPA, TPS, Urokinase plasminogen activator (uPA) and plasminogen activator inhibitor (PAI-1).

The term “additional anticancer agent” includes chemotherapeutic agents selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumurnab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, NO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 345-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. The weight percentage ratio of the anti-cancer exosomes to the one or more excipients can be between about 20:1 to about 1:60, or between about 15:1 to about 1:45, or between about 10:1 to about 1:40, or between about 9:1, 8:1, 7:1, 6:1, 5:1, 4:1, 3:1, 2:1 or 1:1 to about 1:2, 1:3, 1:4, 1:5, 1:6, 1:7, 1:8, 1:9, 1:10, 1:15, 1:20, 1:25, 1:30, or 1:35, and preferably is about 20:1, 19:1, 18:1, 17:1, 16:1, 15:1, 14:1, 13:1, 12:1, 11:1, 10:1, 9:1, 8:1, 7:1, 6:1 or 5:1. In some embodiments, formulations of the invention comprise between about 250 mg to about 500 mg, or between about 300 mg to about 450 mg, or about 325 mg to about 425 mg of total exosomes and may optionally contain one or more suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the pharmaceutical composition comprising anti-cancer exosomes, and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or are apparent to those skilled in the art; for example, see Remington's Pharmaceutical Sciences (17th Ed., Mack Pub. Co. 1985). The composition to be administered will contain a quantity of the selected compound in a pharmaceutically effective amount for therapeutic use in a biological system, including a patient or subject according to the present invention.

Methods of treating patients or subjects in need for a particular disease state or infection comprise administration of an effective amount of a pharmaceutical composition comprising therapeutic amounts of exosomes described herein and optionally at least one additional bioactive (e.g. anti-cancer) agent according to the present invention. The amount of exosomes used in the methods of treatment of the instant invention that may be combined with the carrier materials to produce a single dosage form will vary depending upon the host treated, the particular mode of administration. For example, the compositions could be formulated so that a therapeutically effective dosage of between about 0.01, 0.1, 1, 5, 10, 15, 20, 25, 30, 35, 40, 45, 50, 55, 60, 65, 70, 75, 80, 85, 90 or 100 mg/kg of patient/day or in some embodiments, greater than 100, 110, 120, 130, 140, 150, 160, 170, 180, 190 or 200 mg/kg of the novel exosomes can be administered to a patient receiving these compositions.

Preferably, pharmaceutical compositions in dosage form according to the present invention comprise a therapeutically effective amount of at least 25 mg of exosomes, at least 50 mg of exosomes, at least 60 mg of exosomes, at least 75 mg of exosomes, at least 100 mg of exosomes, at least 150 mg of exosomes, at least 200 mg of exosomes, at least 250 mg of exosomes, at least 300 mg of exosomes, about 350 mg of exosomes, about 400 mg of exosomes, about 500 mg of exosomes, about 750 mg of exosomes, about 1 g (1,000 mg) or more of exosomes, alone or in combination with a therapeutically effective amount of at least one additional anti-cancer agent.

Preferred embodiments of the pharmaceutical compositions of the invention comprise between about 100 mg to about 750 mg, about 250 mg to about 500 mg, or between about 300 mg to about 450 mg, or about 325 mg to about 425 mg, most often about 380 mg of exosomes.

The dose of exosomes administered to a subject can be less than 10 μg, less than 25 μg, less than 50 μg, less than 75 μg, less than 0.10 mg, less than 0.25 mg, less than 0.5 mg, less than 1 mg, less than 2.5 mg, less than 5 mg, less than 10 mg, less than 15 mg, less than 20 mg, less than 50 mg, less than 75 mg, less than 100 mg, less than 500 mg, less than 750 mg, less than 1 g.

The activities of exosomes described herein can be evaluated by methods known in the art, e.g., MTT (3-[4,5-dimehtythiazol-2-yl]-2,5-diphenyltetrazolium bromide) assay, APOPercentage, clonogenic assay, ATP assay, or Extreme Drug Resistance (EDR) assay. See Freuhauf, J. P. and Manetta, A., Chemosensitivity Testing in Gynecologic Malignancies and Breast Cancer 19, 39-52 (1994), which is incorporated by reference in its entirety. The results are then plotted to generate drug response curves, which allow IC₅₀ values (the concentration of a compound required to inhibit 50% of the population of the treated cells) to be determined.

The amount of exosomes required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.01 to about 10 mg/kg of body weight per day.

Other anti-cancer assays are well-known in the art, including in vitro exposure of agents to tumor cells and in vivo antitumor assays in rodent models and rarely, in larger animals.

Purely by way of example, levels of sample cancer cell apoptosis-inducing exosomes and cancer cell sample viability may be correlated with control levels of cancer cell apoptosis-inducing exosomes and cancer cell sample viability by measuring and comparing levels of exosomes and/or levels of normal or cancerous cells, and differences in such levels can range, e.g. from between about 5-10%, or about 10-15%, or about 15-20%, or about 20-25%, or about 25-30%, or about 30-35%, or about 35-40%, or about 40-45%, or about 45-50%, or about 50-55%, or about 55-60%, or about 60-65%, or about 65-70%, or about 70-75%, or about 75-80%, or about 80-85%, or about 85-90%, or about 90-95%, or about 95-100%, or about 100-110%, or about 110-120%, or about 120-130%, or about 130-140%, or about 140-150%, or about 150-160%, or about 160-170%, or about 170-180%, or about 180-190%, or 190-200%, or 200-210%, or 210-220%, or 220-230%, or 230-240%, or 240-250%, or 250-260%, or about 260-270%, or about 270-280%, or about 280-290%, or about 290-300%, or differences of about between about ±50% to about ±0.5%, or about ±45% to about ±1%, or about ±40% to about ±1.5%, or about ±35% to about ±2.0%, or about 30% to about ±2.5%, or about ±25% to about ±3.0%, or about ±20% to about +3.5%, or about +15% to about ±4.0%, or about ±10% to about ±5.0%, or about +9% to about ±1.0%, or about ±8% to about ±2%, or about ±7% to about ±3%, or about +6% to about 5%, or about ±5%, or about ±4.5%, or about 4.0%, or about +3.5%, or about ±3.0%, or about 2.5%, or about ±2.0%, or about +1.5%, or about ±1.0%.

A “biomarker” is any gene or protein whose level of expression in a biological sample is altered compared to that of a pre-determined level. The pre-determined level can be a level found in a biological sample from a normal or healthy subject. Biomarkers include genes and proteins, and variants and fragments thereof. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Biomarkers can be detected, e.g. by nucleic acid hybridization, antibody binding, activity assays, polymerase chain reaction (PCR), S1 nuclease assay and gene chip.

A “control” as used herein may be a positive or negative control as known in the art and can refer to a control cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. For instance, as can be appreciated by a skilled artisan, a control may comprise data from one or more control subjects that is stored in a reference database. The control may be a subject who is similar to the test subject (for instance, may be of the same gender, same race, same general age and/or same general health) but who is known to not have a fibrotic disease. As can be appreciated by a skilled artisan, the methods of the invention can also be modified to compare a test subject to a control subject who is similar to the test subject (for instance, may be of the same gender, same race, same general age and/or same general health) but who is known to express symptoms of a disease. In this embodiment, a diagnosis of a disease or staging of a disease can be made by determining whether protein or gene expression levels as described herein are statistically similar between the test and control subjects.

The terms “level” and/or “activity” as used herein further refer to gene and protein expression levels or gene or protein activity. For example, gene expression can be defined as the utilization of the information contained in a gene by transcription and translation leading to the production of a gene product.

In certain non-limiting embodiments, an increase or a decrease in a subject or test sample of the level of measured biomarkers (e.g. proteins or gene expression) as compared to a comparable level of measured proteins or gene expression in a control subject or sample can be an increase or decrease in the magnitude of approximately ±5,000-10,000%, or approximately ±2,500-5,000%, or approximately ±1,000-2,500%, or approximately ±500-1,000%, or approximately ±250-500%, or approximately 100-250%, or approximately ±50-100%, or approximately ±25-50%, or approximately ±10-25%, or approximately ±10-20%, or approximately ±10-15%, or approximately ±5-10%, or approximately ±1-5%, or approximately ±0.5-1%, or approximately ±0.1-0.5%, or approximately ±0.01-0.1%, or approximately ±0.001-0.01%, or approximately ±0.0001-0.001%.

The values obtained from controls are reference values representing a known health status and the values obtained from test samples or subjects are reference values representing a known disease status. The term “control”, as used herein, can mean a sample of preferably the same source (e.g. blood, serum, tissue etc.) which is obtained from at least one healthy subject to be compared to the sample to be analyzed. In order to receive comparable results the control as well as the sample should be obtained, handled and treated in the same way. In certain examples, the number of healthy individuals used to obtain a control value may be at least one, preferably at least two, more preferably at least five, most preferably at least ten, in particular at least twenty. However, the values may also be obtained from at least one hundred, one thousand or ten thousand individuals.

A level and/or an activity and/or expression of a translation product of a gene and/or of a fragment, or derivative, or variant of said translation product, and/or the level or activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

Certain diagnostic and screening methods of the present invention utilize an antibody, preferably, a monocolonal antibody, capable of specifically binding to a protein as described herein or active fragments thereof. The method of utilizing an antibody to measure the levels of protein allows for non-invasive diagnosis of the pathological states of kidney diseases. In a preferred embodiment of the present invention, the antibody is human or is humanized. The preferred antibodies may be used, for example, in standard radioimmunoassays or enzyme-linked immunosorbent assays or other assays which utilize antibodies for measurement of levels of protein in sample. In a particular embodiment, the antibodies of the present invention are used to detect and to measure the levels of protein present in a sample.

Humanized antibodies are antibodies, or antibody fragments, that have the same binding specificity as a parent antibody, (i.e., typically of mouse origin) and increased human characteristics. Humanized antibodies may be obtained, for example, by chain shuffling or by using phage display technology. For example, a polypeptide comprising a heavy or light chain variable domain of a non-human antibody specific for a disease related protein is combined with a repertoire of human complementary (light or heavy) chain variable domains. Hybrid pairings specific for the antigen of interest are selected. Human chains from the selected pairings may then be combined with a repertoire of human complementary variable domains (heavy or light) and humanized antibody polypeptide dimers can be selected for binding specificity for an antigen. Techniques described for generation of humanized antibodies that can be used in the method of the present invention are disclosed in, for example, U.S. Pat. Nos. 5,565,332; 5,585,089; 5,694,761; and 5,693,762. Furthermore, techniques described for the production of human antibodies in transgenic mice are described in, for example, U.S. Pat. Nos. 5,545,806 and 5,569,825.

In order to identify small molecules and other agents useful in the present methods for treating a cancer by modulating the activity and expression of a disease-related protein and biologically active fragments thereof can be used for screening therapeutic compounds in any of a variety of screening techniques. Fragments employed in such screening tests may be free in solution, affixed to a solid support, borne on a cell surface, or located intracellularly. The blocking or reduction of biological activity or the formation of binding complexes between the disease-related protein and the agent being tested can be measured by methods available in the art.

Other techniques for drug screening which provide for a high throughput screening of compounds having suitable binding affinity to a protein, or to another target polypeptide useful in modulating, regulating, or inhibiting the expression and/or activity of a disease, are known in the art. For example, microarrays carrying test compounds can be prepared, used, and analyzed using methods available in the art. See, e.g., Shalon, D. et al., 1995, International Publication No. WO95/35505, Baldeschweiler et al., 1995, International Publication No. WO95/251116; Brennan et al., 1995, U.S. Pat. No. 5,474,796; Heller et al., 1997, U.S. Pat. No. 5,605,662.

Identifying small molecules that modulate protein activity can also be conducted by various other screening techniques, which can also serve to identify antibodies and other compounds that interact with proteins identified herein and can be used as drugs and therapeutics in the present methods. See, e.g., Enna et al., eds., 1998, Current Protocols in Pharmacology, John Wiley & Sons, Inc., New York N.Y. Assays will typically provide for detectable signals associated with the binding of the compound to a protein or cellular target. Binding can be detected by, for example, fluorophores, enzyme conjugates, and other detectable labels well known in the art. The results may be qualitative or quantitative.

For screening the compounds for specific binding, various immunoassays may be employed for detecting, for example, human or primate antibodies bound to the cells. Thus, one may use labeled anti-hIg, e.g., anti-hIgM, hIgG or combinations thereof to detect specifically bound human antibody. Various labels can be used such as radioisotopes, enzymes, fluorescers, chemiluminescers, particles, etc. There are numerous commercially available kits providing labeled anti-hlg, which may be employed in accordance with the manufacturer's protocol.

In one embodiment, a kit can comprise: (a) at least one reagent which is selected from the group consisting of (i) reagents that detect a transcription product of the gene coding for a protein marker as described herein (ii) reagents that detect a translation product of the gene coding for proteins, and/or reagents that detect a fragment or derivative or variant of said transcription or translation product; (b) optionally, one or more types of cells, including engineered cells in which cellular assays are to be conducted; (c) instructions for diagnosing, or prognosticating a disease, or determining the propensity or predisposition of a subject to develop such a disease or of monitoring the effect of a treatment by determining a level, or an activity, or both said level and said activity, and/or expression of said transcription product and/or said translation product and/or of fragments, derivatives or variants of the foregoing, in a sample obtained from said subject; and comparing said level and/or said activity and/or expression of said transcription product and/or said translation product and/or fragments, derivatives or variants thereof to a reference value representing a known disease status (patient) and/or to a reference value representing a known health status (control) and/or to a reference value; and analyzing whether said level and/or said activity and/or expression is varied compared to a reference value representing a known health status, and/or is similar or equal to a reference value representing a known disease status or a reference value; and diagnosing or prognosticating a disease, or determining the propensity or predisposition of said subject to develop such a disease, wherein a varied or altered level, expression or activity, or both said level and said activity, of said transcription product and/or said translation product and/or said fragments, derivatives or variants thereof compared to a reference value representing a known health status (control) and/or wherein a level, or activity, or both said level and said activity, of said transcription product and/or said translation product and/or said fragments, derivatives or variants thereof is similar or equal to a reference value and/or to a reference value representing a known disease stage, indicates a diagnosis or prognosis of a disease, or an increased propensity or predisposition of developing such a disease, a high risk of developing signs and symptoms of a disease.

Reagents that selectively detect a transcription product and/or a translation product of the gene coding for proteins can be sequences of various length, fragments of sequences, antibodies, aptamers, siRNA, microRNA, and ribozymes. Such reagents may be used also to detect fragments, derivatives or variants thereof.

Exosomes may be loaded with small molecules, antisense oligonucleotides, siRNAs, peptides, proteins or antibodies that target, e.g. HER2, ERalpha, BRCA1, BRCA2, EGFR1, PIK3CA, PTEN, TP53, RB or other breast cancer oncogenes or oncogene translation products. For example, RNA silencing agents (including siRNA) as described in United States Patent Application Document No. 20140356350 are examples of breast cancer therapeutics that can be loaded into exosomes of the invention.

In certain embodiments, exosomes of the invention are loaded with antibodies directed against the HER2 extracellular domain (e.g. trastuzumab (Herceptin®), antibodies that inhibit the homodimerization and/or heterodimerization of HER2 (e.g. pertuzumab), anti-HER2 vaccines, inhibitors of HER2 tyrosine kinase activity (e.g. emodin (3-methyl-1,6,8-trihydroxyanthraquinone), curcumin, OSI-774 (Tarceva®), ZD-1839 (Iressa®), CI-1033 and lapatinib (Tykerb®), intracellular single-chain antibodies directed against HER2, inhibitors of transcription of the gene coding for HER2 (e.g. adenovirus E1A gene) or inhibitors of HER2 mRNA translation (e.g. antisense oligonucleotides and ribozymes).

In certain other embodiments, exosomes of the invention are loaded with anti-estrogens and selective estrogen receptor modulators (SERMs), e.g. tamoxifen, raloxifene, droloxifene, 4-hydroxytamoxifen, trioxifene, keoxifene, LYI17018, onapristone, toremifene, aromatase inhibitors (e.g. 4(5)-imidazoles, aminoglutethimide, megestrol acetate, exemesiane, formesianic, fadrozole, vorozole, letrozole, anastrozole), anti-androgens (e.g. flutamide, nilutamide, bicalutamide, leuprolide, goserelin, troxacitabine, antisense oligonucleotides (e.g. PKC-alpha, Raf, H-Ras, and epidermal growth factor receptor (EGF-R), vaccines including gene therapy vaccines (e.g. ALLOVECTIN, LEUVECTIN, and VAXID, PROLEUKIN or rIL-2, LURTOTECAN or topoisomerase 1 inhibitor, ABARELIX or rmRH, and/or calicheamicin, maytansinoids, dolastatins, aurostatins, a trichothecene, and CC1065.

As used herein “apoptosis” means cell death mediated by the apoptotic pathway and measured, e.g. by caspase 3/7 activity.

As used herein “cytotoxicity” means a measure of cell death quantified by cell membrane permeability to the CeilTox fluorescent reagent (Promega).

As used herein “exosome” means an extracellular vesicle of about 20 nm-250 nm in size consisting of fluid, macro-molecules, solutes, and metabolites contained by a lipid bilayer or micelle. The term “autologous exosomes is used to describe a population of exosomes which are obtained from a subject or patient to whom the exosomes are to be administered.

The term “effective” means an amount of concentration which is used to effect an intended result. In the case of exosomes which are used to treat cancer, including a cancerous tumor, an effective amount of exosomes (synonymous with a “therapeutically effective amount”) to treat a tumor is an anticancer effective amount within the range of about 0.1 μg to about 100 mg (often about 0.5 μg to about 50 mg, about 1 μg to about 25 mg within the broader range) per ml (mg) of tumor to be treated.

As used herein “field cancerized tissue” means histologically normal tissue surrounding a tumor mass that demonstrates molecular alterations characteristic of cancer cells.

As used herein “medium”, “media”, means the fluid in which the cells are cultured in, containing nutrients essential for cell growth. “Conditioned media” and “CM” mean media in which cells have been growing in for a defined amount of time.

As used herein neoplastic cancer means an abnormal cell growth containing cancer cells.

As used herein “tumorigenic” means a property that gives rise to a tumor

Exosomes are small (about 20-250 nanometers in diameter) vesicles secreted by most cell types. Exosomes secreted by fibroblasts derived from Tumor Adjacent Histologically Normal tissue 1 cm, 3 cm and 5 cm from the tumor (TAHN-1, TAHN-3, TAHN-5, respectively) have been shown to have different effects relating to the proliferation, cell membrane permeability and apoptosis of malignant and non-malignant breast epithelial cells.

Exosome-based cancer therapy can be used alone or in combination with a chemotherapeutic as a therapeutic for neoplastic cancer and tumors such as occur in the breast, prostate, pancreas and other organs. Because exosomes are a natural method of cell communication in the human body, their mechanism of targeting and eliminating cancer cells is highly controlled and involves multi-factorial targets, potentially making drug resistance significantly less likely.

Exosome research has been focused on utilizing exosomes as cancer diagnostic and prognostic tools. Utilizing exosomes as a therapeutic agent is a relatively new area of exploration. Other researchers' current investigations on exosomes as therapeutic agents have focused on immune-cell-derived exosomes as a mechanism to manipulate the immune response to cancer. Our approach is novel in exploiting the direct cancer-fighting properties of specific exosomes which are not related to immune cells or the immune response.

Exosome-based cancer therapy offers two significant advantages over both chemotherapeutic agents and targeted agents currently used in clinical practice:

### 1. High Specificity.

As currently used chemotherapeutic agent target broad biological processes, such as DNA replication. Although agents are more cytotoxic to cancer cells, normal cells also experience varying degrees of cytotoxicity, dependent on cell type. We have demonstrated that TAHN exosomes are cytotoxic to two breast cancer cell lines, while leaving non-malignant cells unaffected. This specificity is likely to result in little to no side effects.

### 2. Decreased Risk of Drug Resistance.

Due to the possibility for clinically available targeted therapies to target single molecules or pathways, selection of cell populations lacking the target can occur, and eventual drug resistance. Because exosomes are a natural method of cell communication in the human body, their mechanism of targeting and eliminating cancer cells has evolved to address multi-faceted targets, potentially making resistance less likely. In accordance with the present invention, exosomes can be obtained from any suitable cell type as discussed above, or by isolation from physiological fluids. Typically, the methods of the present invention comprise isolation of the exosomes from cell culture medium or tissue supernatant.

As described in U.S. Patent Application Document No. 20140356382, “[e]xosomes produced from cells can be collected from the culture medium by any suitable method. Typically a preparation of exosomes can be prepared from cell culture or tissue supernatant by centrifugation, filtration or combinations of these methods. For example, exosomes can be prepared by differential centrifugation, that is low speed (<2,0000 g) centrifugation to pellet larger particles followed by high speed (>100,000 g) centrifugation to pellet exosomes, size filtration with appropriate filters (for example, 0.22 .mu. m filter), gradient ultracentrifugation (for example, with sucrose gradient) or a combination of these methods.”

Further, as described in U.S. Patent Application Document No. 20140356382, “exogenous protein and/or peptide can be introduced into the exosomes by a number of different techniques [including] electroporation or the use of a transfection reagent. [I]t is possible to use electroporation to load exosomes with antibodies. Electroporation conditions may vary depending on the charge and size of the biotherapeutic cargo. Typical voltages are in the range of 20V/cm to 1,000V/cm, such as 20V/cm to 100V/cm with capacitance typically between 25 μF and 250 g, such as between 25 μF and 125 μf. A voltage in the range of 150 mV to 250 mV, particularly a voltage of 200 mV is preferred for loading exosomes with an antibody . . . . Alternatively, the exosomes may be loaded with exogenous protein and/or peptide using a transfection reagent. Despite the small size of the exosomes, conventional transfection agents may be used for transfection of exosomes with protein and/or peptide. [E]xosomes may also be loaded by transforming or transfecting a host cell with a nucleic acid construct which expresses therapeutic protein or peptide of interest, such that the therapeutic protein or peptide is taken up into the exosomes as the exosomes are produced from the cell.

Exosomes produced from cells can be collected from the culture medium by any suitable method. Typically a preparation of exosomes can be prepared from cell culture or tissue supernatant by centrifugation, filtration or combinations of these methods. For example, exosomes can be prepared by differential centrifugation, that is low speed (<2,0000 g) centrifugation to pellet larger particles followed by high speed (>100,000 g) centrifugation to pellet exosomes, size filtration with appropriate filters (for example, 0.22 μm filter), gradient ultracentrifugation (for example, with sucrose gradient) or a combination of these methods.” Id.

In illustrative embodiments, TAHN cells, e.g. TAHN-5 cells, are cultured for about 1, 2, 3, 4, 5, 6 or 7 days, or for as long as about 1, 2, 3, 4, 5, 6, 7, 8 weeks or about 1, 2, 3, 4, 5, or 6 months. The TAHN cells (TAHN-5 cells) may be cultured in suitable media and grown under conditions that are readily determined by one of ordinary skill in the art. Cell culture conditions may vary with cell type and the examples presented hereinafter illustrate suitable media and conditions. Alternatively, CMRL 1066 medium (from Invitrogen) with fetal bovine serum (e.g., at 10%) and optionally supplemented with glutamine or glutamine-containing mixtures and antibiotics could be used. Cells can be grown on a surface in some embodiments, e.g. they can be grown as a monolayer on the surface and may be grown until 50, 60, 70, 80, 90, 95 or 100% confluent.

Exosomes can be harvested at various time intervals (e.g. at about 2, 4, 6, 8 or 3, 6, 9 or 12 day intervals). Exemplary yields of exosomes can range about 0.2 μg exosomes/1 million TAHN-5 cells, at least about 0.3 μg g exosomes/1 million TAHN-5 cells, at least about 0.4 μg g exosomes/1 million TAHN-5 cells, at least about 0.5 μg g exosomes/1 million TAHN-5 cells, at least about 0.6 μg g exosomes/1 million TAHN-5 cells, at least about 0.7 μg g exosomes/1 million TAHN-5 cells, at least about 0.8 μg g exosomes/1 million TAHN-5 cells, at least about 0.9 μg g exosomes/1 million TAHN-5 cells, at least about 1.0 μg g exosomes/1 million TAHN-5 cells, at least about 1.5 μg g exosomes/1 million TAHN-5 cells, at least about 2.0 μg g exosomes/1 million TAHN-5 cells, at least about 2.5 μg g exosomes/1 million TAHN-5 cells, at least e.g. about 3.0 μg g exosomes/1 million TAHN-5 cells, at least about 5.0 μg g exosomes/1 million TAHN-5 cells, and at least about 10.0 .mu. g exosomes/1 million TAHN-5 cells, during a time period of about 48 hours of culture of TAHN-5 cells.

In certain embodiments, exosomes are harvested and collected by ultracentrifugation or differential centrifugation or any combination thereof, pelleted exosomes are collected, and, optionally, collected pelleted exosomes are washed with a suitable medium.

“Substantially purified exosomes” means exosomes that are approximately 50%, 55%, 60%, 65%, 70%, 75%, 80%, 90%, 91%, 92%, 93%, 94%, 95%, 96%, 97%, 98%, 99% or 100% free of any other component found in the cultured medium from which the exosomes were harvested.

Preferred intravenous formulations of the invention comprise the substantially purified exosomes described herein, an isotonic medium and one or more substances preventing aggregation of the exosomes. Preferred intravenous formulations may therefore contain saline solutions (e.g. normal saline (NS); about 0.91% w/v of NaCl, about 300 mOsm/L) and/or dextrose 4% in 0.18% saline, and optionally 1%, 2% or 3% human serum albumin.

In exemplary embodiments, preferred intravenous formulations of the invention may comprise about 0.1 μg exosomes/ml medium, about 0.2 μg exosomes/ml intravenous medium, about 0.3 μg exosomes/ml intravenous medium, about 0.4 μg exosomes/ml intravenous medium, about 0.5 μg exosomes/ml intravenous medium, about 0.6 μg exosomes/ml intravenous medium, about 0.7 μg exosomes/ml intravenous medium, about 0.8 exosomes/ml intravenous medium, about 0.9 μg exosomes/ml intravenous medium, about 1.0 μg exosomes/ml intravenous medium, about 1.5 μg exosomes/ml intravenous medium, about 2.0 μg exosomes/ml intravenous medium, about 2.5 μg exosomes/ml intravenous medium, such as at least e.g. about 3.0 μg exosomes/ml intravenous medium, such as e.g. at least about 5.0 μg exosomes/ml intravenous medium, about 10.0 μg exosomes/ml intravenous medium, 15.0 μg exosomes/ml intravenous medium or about 20.0 μg or more exosomes/ml intravenous medium.

These and other aspects of the invention are illustrated further in following non-limiting examples.

### Example 1

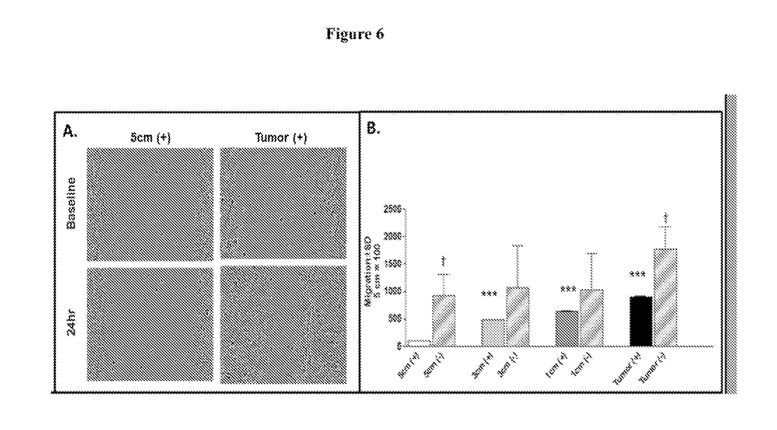

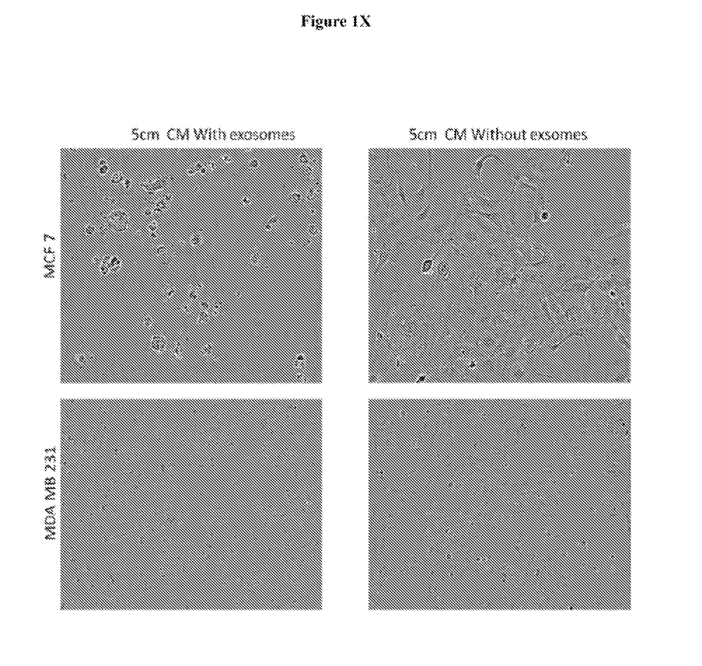

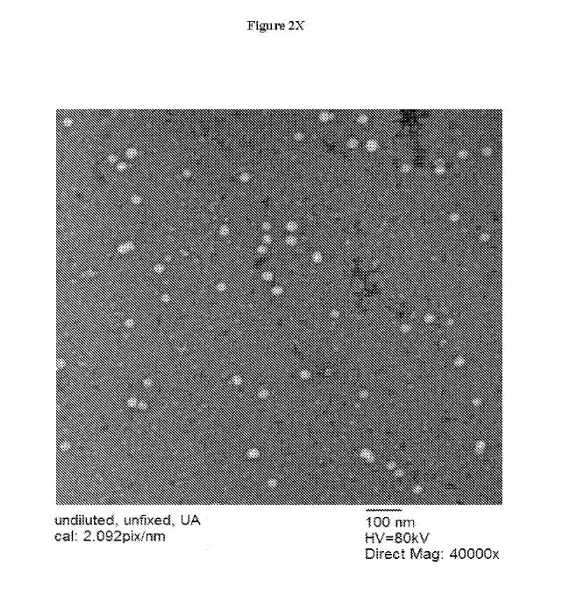

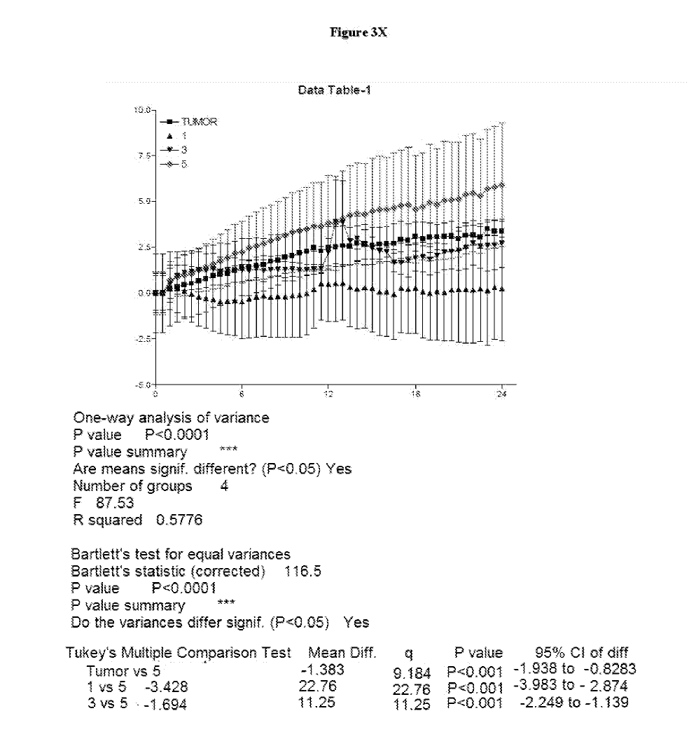

One of our most striking observations is the similarity of FCT fibroblasts to CAFs in their ability to induce tumorigenic properties in neighboring epithelia. Interestingly, we have shown that this effect is not due to a factor produced by FCT fibroblasts and CAFs, as originally predicted. Rather, this effect is due to their lack of a factor produced by patient-matched normal fibroblasts further from the tumor that suppresses tumorigenic properties. We have demonstrated that conditioned media (CM) from TAHN-5 fibroblasts inhibits migration of normal breast epithelial cells, but does not affect their viability (FIG. 6A). Moreover, CAF or TAHN-1 fibroblast conditioned media does not demonstrate this inhibition. We have also demonstrated that the factor causing the inhibition can be removed from the conditioned media by centrifugation at 10,000×g for 45 minutes-conditions typically used to pellet exosomes (FIG. 6B).

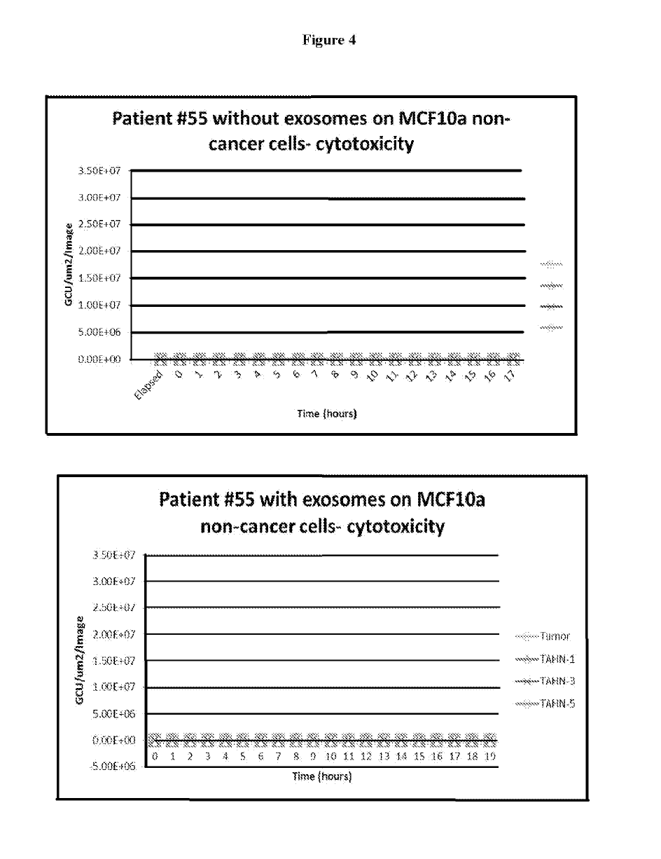

In addition to inhibition of migration of normal breast epithelial cells, the TAHN-5 fibroblast conditioned media with exosomes induces significant cell death in both MCF7 and MDA-MB231 breast cancer cells. We have demonstrated this phenomenon morphologically (FIG. 1X). CM from tumor fibroblasts, or reduction mammoplasty fibroblasts do not have the ability to cause this phenomenon. FIG. 2X is shows the results of electron microscopy of exosomes derived from TAHN-5 fibroblasts. FIG. 3X provides preliminary data showing that a statistically significant higher amount of apoptosis took place in the TAHN-5 fibroblast population. To demonstrate this, we plated MDA-MB 231 breast cancer cells in 96-well plates overnight. We then treated with Conditioned media with and without exosomes from 3 patient sample sets of tumor, TAHN-1, TAHN-3 and TAHN-5 fibroblasts. At this time we also added the Cell Player Caspase 3/7 reagent to monitor for apoptosis. Images were obtained and analyzed using the IncuCyte Zoom green fluorescent module. Mean fluorescence/object is plotted in FIG. 4X. All statistics were performed using the Prism Graph Pad Statistical Package.

### Example 2

Exosomes secreted by TAHN-5 fibroblasts demonstrate cancer-specific cytotoxicity in vitro. TAHN-5 fibroblast exosomes selectively induce apoptosis in MDA-MB231 and MCF7 malignant breast cell lines, but not in MCF10a non-malignant cells. One embodiment of the present invention provides for the therapeutic use of exosomes derived from the histologically normal tissue of the cancer affected organ but located outside of a field cancerized tissue, for example TAHN tissue.

Tissue adjacent to breast tumors, although histologically normal, possesses many of the molecular abnormalities found in patient matched tumor tissues. The epithelial cells demonstrate evidence of “Hallmarks of Cancer”, such as genomic instability⁸, re-activation of telomerase^{9,10} and epithelial to mesenchymal transition¹¹. The fibroblasts exhibit wound healing gene expression, secretion of dense extracellular matrix, and the ability to contract¹¹. These are properties of Carcinoma Associated Fibroblasts (CAFs) which are known to promote tumorigenesis in adjacent cells. These alterations decrease as a function of distance from the tumor. We term tumor adjacent tissue Tumor Adjacent Histologically Normal tissue. The TAHN tissue specimens from breast cancer-affected mammary organs used in our experiments so far were collected about 1 cm, 3 cm and 5 cm (+/−1 cm) from the tumor and thus were dubbed TAHN-1, TAHN-3, TAHN-5, respectively. However, they ranged in size, and therefore the tissue within a specimen may contain tissue from less than the desired measured location and some tissue at a distance from the tumor margin that is greater than the desired measured location. Embodiments of the present invention relate to exosomes produced by fibroblast populations within TAHN tissue, further selected by size, as well as by their cancer-cell specific cytotoxicity and/or ability to cause apoptosis of cancer cells.

Conditioned Media from TAHN Fibroblasts Secrete Exosomes

We investigated if TAHN fibroblasts secrete exosomes. Conditioned Media (CM) was collected from primary cell cultures of fibroblasts derived from tumor, TAHN-1, TAHN-3 and TAHN-5 tissues. Exosomes were collected via differential centrifugation, and the potential exosome-containing pellet was imaged with electron microscopy. FIG. 1 shows representative images of tumor, TAHN-1 and TAHN-5 exosomes. Size analysis showed differences in the distribution of exosome size that was dependent on distance from the tumor (FIG. 1).

Exosomes from TAHN-3 and TAHN-5 Fibroblasts Suppress Proliferation in Malignant Breast Cell Lines.

Knowing that FCT fibroblasts secreted exosomes, we evaluated the effect of these exosomes on the proliferation of malignant breast and non-malignant cells using Live Content Imaging (Incucyte Zoom, Essen Biosciences). We treated malignant MCF7 and non-malignant MCF10a cells with fibroblast conditioned media (CM) from three patients with and without exosomes. The CM was derived from primary fibroblast cultures from three patient samples of tumor, TAHN-1, TAHN-3 and TAHN-5 tissues. Fibroblasts from TAHN-3 and TAHN-5 tissue secreted exosomes that inhibited the proliferation of the breast cancer cells. A difference of least squares means analysis demonstrated that malignant MCF7 cells treated with CM from TAHN-1 fibroblasts with exosomes had proliferation rates similar to those treated with tumor fibroblasts CM (dashed line, FIG. 2a). These rates were not significantly different than non-treated MCF7 cells. However, both TAHN-3 and TAHN-5 CM significantly reduced the levels of proliferation (FIG. 2a). Removal of the exosomes eliminated the effect (FIG. 2b). A difference of least squares means analysis demonstrated that the same CM did not have a significant effect on non-malignant MCF10a cells (data not shown).

Exosomes Derived from TAHN-5 Exosomes Induce Cytotoxicity and Apoptosis in Malignant Cells.

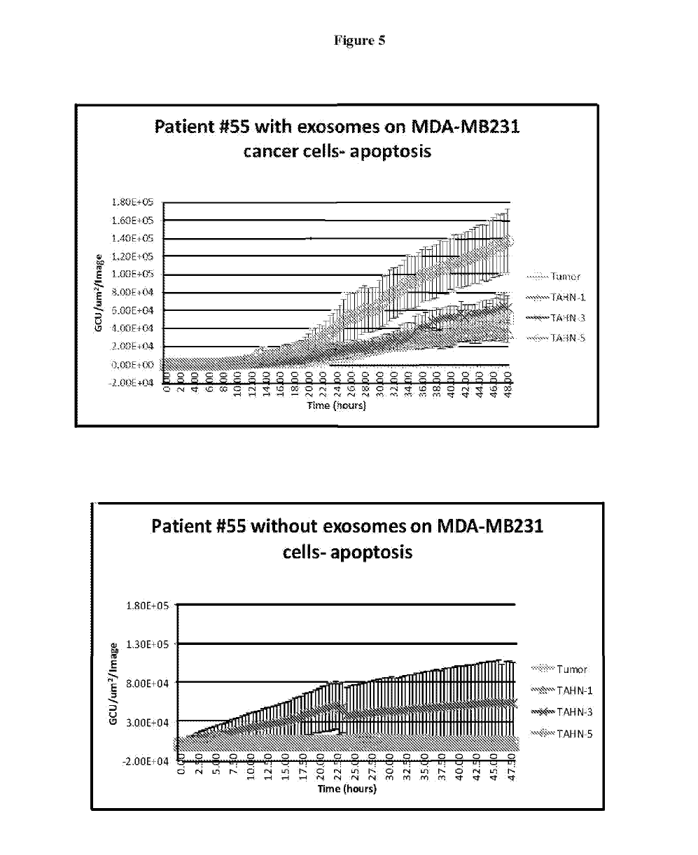

To determine if the reduction in proliferation was in part due to cell death, we tested the effect of TAHN fibroblast exosomes on cytotoxicity and apoptosis of malignant breast cells. We treated malignant MDA-MB231 cells with CM with and without exosomes from TAHN-1, TAHN-3, TAHN-5 and tumor fibroblasts from 3 patients. We monitored cytotoxicity with the CellTox Assay (Promega) and monitored green fluorescence in real time for 24 hours in the IncuCyte Zoom instrument. As shown in FIG. 3a, cytotoxicity was significantly higher in cells treated with TAHN-5 CM, than any other CM. The effect was lost upon removal of exosomes (FIG. 3b). Cytotoxicity was shown to be in part due to apoptosis (FIGS. 5a and 5b) Cytotoxicity was not observed in non-malignant MCF10a cells when treated with the same conditioned media (FIGS. 4a and 4b).

Another aspect of the present invention is to produce exosomes with enhanced efficacy by treating the cells producing the exosomes with drugs that increase the cytotoxicity of the exosomes with regard to tumor cells. For example, exosomes that produce apoptosis of tumor cells >100,000 GCU/um2/Image and/or exosomes that produce cytotoxicity of tumor cells >17,500,000 GCU/um2/Image in the IncuCyte Zoom Live Content Imaging instrument are selected. GCU is Green Calibrated Unit.

To perform the above listed experiments, tumor and TAHN tissue from mastectomy surgeries were excised from tumor and tumor-adjacent tissue at defined distances (1 cm, 3 cm and 5 cm) from the visible tumor margin. Tissues were stored in Dulbecco's modified Eagle's medium (DMEM) supplemented with 200 U/ml penicillin and 200 μg/ml streptomycin until processing (typically within 1-2 hours of surgery). Half of each sample was snap frozen, sectioned and characterized histologically. The remaining half of the sample was used to establish primary cultures. Short term primary cultures of mammary cells were derived from “organoid” preparations of breast tissues, as previously described^{18,38}. Briefly, tissue samples were minced and enzymatically dissociated using 0.1% w/v collagenase I in Mammary epithelial growth medium (MEGM, Lonza) at 37° C. for 12-18 hrs. Small tissue fragments (organoids) remaining after digestion were collected by centrifugation at 100×g for 2 min. These organoids were seeded directly into DMEM supplemented with 10% FBS. Differential trypsinization and differential centrifugation were performed for maintenance of the fibroblast population.

Preparation of Conditioned Media—

Fibroblasts from tumor (positive control), patient-matched TAHN-1, TAHN-3, TAHN-5 and non-patient matched tissue from a reduction mammoplasty surgery (cancer-negative control) were grown to confluence, at which point media was replaced. 24 hours later, conditioned media was removed, filtered through a 2 μm filter and stored at 4 C as described²¹. Isolation of exosomes was performed by sequential ultracentrifugation at 2,000×g for 30 min, 10,000×g for 40 min, and 100,000×g for 2-14 hr. Exosomes were washed with PBS, and purified by centrifugation at 100,000×g for 2 hr³⁹.

The Effect of Conditioned Media from Fibroblast Populations on Malignant MDA-MB231 Cells.

We will initially perform optimization experiments. This will include adapting the MDA-MB231 cells to grow in a 384-well plate and optimizing the number of cells per well. We will optimize the assay volume based on the duration of incubation. Following optimization, we will grow MDA-MB 231 cells in conditioned media from fibroblasts derived from TAHN cultures with and without exosomes in triplicate. Following growth in conditioned media, cytotoxicity will be measured in real time in the IncuCyte Zoom instrument using the CellTox Green Assay (Promega). CeilTox object counts/mm² will be measured over time using IncuCyte Zoom's basic analyzer. Area under the curve of CellTox™ object counts/mm² over time will be used to determine level of cytotoxicity. We will rank all tested fibroblast populations based on cytotoxicity. We will select the fibroblasts that produce the 2 most cytotoxic and 2 least cytotoxic exosomes for treatment with drugs.

### Experimental Protocol

### Processing Tissue:

The Digestion Medium s—10 mL Fibroblast Media and 100 μL Collagenase A.

The Culture Medium:—Fibroblast is 500 mL DMEM, 50 mL FBS-HI, 5 mL PenStrep

The Culture Medium—HMEC is 500 mL MEBM, 1 Lonza MEGM Bullet kit.

First the tissue of interest is chopped into a fine puree. Then the chopped tissue is transferred into a 15 mL conical tube containing the Digestion Medium. Next the digestion Medium is set on the rocker in the non-CO2 incubator for 18 hours or until the suspension has a “smoothie” texture with no large chunks or pieces remaining. This suspension is spun down in the conical tubes for 10 min at 1400 rpm. The top layer of the fat is aspirated leaving behind the supernatant. The remaining supernatant is transferred to a new 15 mL conical tube labeled “supernatant”. The supernatant is spun down for 10 min at 1600 rpm. Aspirate the supernatant and resuspend the remaining pellet in Fibroblast Medium. Plate this pellet in a 6-well plate labeled appropriately.

Divide half of the pellet for HMEC growth and half for Fibroblast growth. Resuspend the pellet in the 600 μL of medium. Deposit the cells onto the plate surface drop by drop using a 1 mL micro-pipet. Do not add additional media. Gently add 1 mL of either Fibroblast medium or HMEC medium to each well the following morning.

### Maintaining and Passaging Primary Cells:

First, collect Fibroblast conditioned media into a 15 mL conical tube and store in deli fridge. Aspirate HMEC media. Next rinse a 6-well with 1 mL PBS/well or 2 mL PBS for T-25 flask. Aspirate PBS.

Trypsinize cell using 1:4 Trypsin:PBS w/out Calcium. Use 1 mL diluted trypsin/well in 6-well plate or 2 mL diluted trypsin for T-25 flask. Place in incubator and check every 4 minutes until the cells have lifted. Organoids should remain adhered to the vessel. Neutralize trypsin using TNS 1:2-1 diluted trypsin:2 TNS. Then spin the cells for 10 minutes at 900 rpm. Thereafter gently rinse the organoids that are still adhered to the vessel with PBS (twice for HMEC to remove all FBS). Aspirate the PBS. Add new media and place back in the incubator. The supernatant is aspirated and the cells are resuspended in new Fibroblast or HMEC medium and plated in new vessels. (If a partial trypsinization was done I label as PT+1:P#→first partial tryspinization: Passage #.). For cell maintenance the old media is aspirated and washed with PBAS, then fresh media is added.

### Isolating Exosomes:

Gently pipette the conditioned media to resuspend any settled exosomes. The conditioned media is passed through 0.2 μL syringe filters and the filters are washed to collect exosomes. The filtered conditioned media is spun at 10,700 RPM at 4° C. for 1 hour. Thereafter the supernatant is removed to not disturb the pellet. This supernatant is exosome free. The exosome pellet is used for treatment or stored in PBS w/ions at 4° C.

### NMR Spectroscopy of Exosomes.

### Tissue Preparation

Breast tumor and matched adjacent histologically-normal tissue from 3 cm and 5 cm from the tumor margin were collected from one patient from the UNM-HSC Human Tissue Repository as approved by federal guidelines. Upon arrival, tissues were rinsed with Dulbecco's PBS, supplemented with antibacterial and antimycotic agents (200 U/ml penicillin, 200 μg/ml streptomycin, 5 μg/ml amphotericin B). Tissues were physically separated by mincing, followed by enzymatic disaggregation via treatment with 0.1% collagenase I for 16-36 hours at 37° C. (1 mg/ml collagenase I, 100 U/ml penicillin, 100 μg/ml streptomycin in Dulbecco's modified Eagle's medium (DMEM)). After this incubation, cells were rinsed with PBS and human mammary epithelial medium (DMEM supplemented with 2 mM glutamine, 100 U/ml penicillin, 100 μg/ml streptomycin, 10 mM Hepes, 0.075% bovine serum albumin, 0.5 μg/ml hydrocortisone, 5 μg/ml insulin, 5 ng/ml EGF).

### Fibroblast Growth

Small tissue fragments (organoids) remaining after digestion were collected by centrifugation at 100×g for 2 min. These organoids were seeded directly into DMEM supplemented with 10% FBS. Differential trypsinization and differential centrifugation were performed for maintenance of the fibroblast population. Fibroblasts from tumor, patient-matched normal adjacent tissue 3 cm and 5 cm from the tumor margin were grown to confluence, at which point media was replaced. 24 hours later, conditioned media was removed and stored at 4 C as described. See, Luga, et al., Cell 2012; 151:1542-56.

### Exosomes Isolation-Ultracentrifugation Protocol

Isolation of exosomes was performed by sequential ultracentrifugation at 2,000×g for 30 min, 10,000×g for 40 min, and 100,000×g for 2-14 hr. Exosomes were washed with PBS, and purified by centrifugation at 100,000×g for 2 hr. See, Thery, C, Amigorena S, Raposo G, Clayton A. “Isolation and characterization of exosomes from cell culture supernatants and biological fluids”. Current protocols in cell biology/editorial board, Juan S. Bonifacino . . . [et al.] 2006; Chapter 3:Unit 3 22.

### NMR Spectroscopy

The isolated exosomes were resuspended and placed into 0.5 mL deuterated phosphate buffered saline at pH 7.4 and transferred to 5 mm NMR tubes. One microliter of a 50 mM solution of deuterated disilapentane sulfonate was added to provide an internal chemical shift reference. NMR spectra were obtained at 300 MHz with the aid of a Bruker Avance300 NMR system using a 6 kHz sweep width collected into 4K data points following a 7 microsecond (90 degree) pulse with an acquisition time of 0.58 sec, and a recycle time of 2 seconds. The time domain spectra after 256 transients were filtered with a 3 Hz exponential and Fourier transformed. The resulting spectra showed the expected signals from the lipid vesicle portion of the exosomes, with peaks at characteristic frequencies indicative of phospholipids: 1.3 ppm (—CH₂—)ₙ, and 0.89 ppm (—CH₃). These signals also had large widths (˜150 Hz) indicative of closely-packed fatty-acyl chains in phospholipid bilayers and consistent with the expected properties of the exosomes.

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited herein are hereby incorporated by reference.

### REFERENCES

1. Azmi, A. S., Bao, B. & Sarkar, F. H. Exosomes in cancer development, metastasis, and drug resistance: a comprehensive review. Cancer Metastasis Rev 32, 623-642 (2013).
2. Kahlert, C. & Kalluri, R. Exosomes in tumor microenvironment influence cancer progression and metastasis. Journal of molecular medicine 91, 431-437 (2013).
3. Chen, W., et al. Efficient induction of antitumor T cell immunity by exosomes derived from heat-shocked lymphoma cells. European journal of immunology 36, 1598-1607 (2006).
4. Li, W., et al. Exosomes derived from Rab27aoverexpressing tumor cells elicit efficient induction of antitumor immunity. Molecular medicine reports 8, 1876-1882 (2013).
5. Heaphy, C. M., et al. Telomere DNA content and allelic imbalance demonstrate field cancerization in histologically normal tissue adjacent to breast tumors. Int J Cancer 119, 108-116 (2006).
6. Meeker, A. K. & Argani, P. Telomere shortening occurs early during breast tumorigenesis: a cause of chromosome destabilization underlying malignant transformation? J Mammary Gland Biol Neoplasia 9, 285-296 (2004).
7. Meeker, A. K., et al. Telomere length abnormalities occur early in the initiation of epithelial carcinogenesis. Clin Cancer Res 10, 3317-3326 (2004).
Kim, N. W., et al. Specific association of human telomerase activity with immortal cells and cancer. Science 266, 2011-2015 (1994).
9. Yang, G., et al. Knockdown of p53 combined with expression of the catalytic subunit of telomerase is sufficient to immortalize primary human ovarian surface epithelial cells. Carcinogenesis 28, 174-182 (2007).
10. Hines, W. C., Fajardo, A. M., Joste, N. E., Bisoffi, M. & Griffith, J. K. Quantitative and spatial measurements of telomerase reverse transcriptase expression within normal and malignant human breast tissues. Mol Cancer Res 3, 503-509 (2005).
11. Trujillo, K. A., et al. Breast Field Cancerization: Isolation and Comparison of Telomerase Expressing Cells in Tumor and Tumor Adjacent, Histologically Normal Breast Tissue. Mol Cancer Res (2011).
12. Trujillo, K. A., et al. Markers of fibrosis and epithelial to mesenchymal transition demonstrate field cancerization in histologically normal tissue adjacent to breast tumors. Int J Cancer (2010).
13. Schafer, M. & Werner, S. Cancer as an overhealing wound: an old hypothesis revisited. Nat Rev Mol Cell Biol 9, 628-638 (2008).
14. Troester, M. A., et al. Activation of host wound responses in breast cancer microenvironment. Clin Cancer Res 15, 7020-7028 (2009).
15. Yu, Y., et al. Cancer-associated fibroblasts induce epithelial-mesenchymal transition of breast cancer cells through paracrine TGF-beta signalling. Br J Cancer (2013).
16. Luga, V., et al. Exosomes mediate stromal mobilization of autocrine Wnt-PCP signaling in breast cancer cell migration. Cell 151, 1542-1556 (2012).
1A. Heaphy, C. M., et al. Telomere DNA content and allelic imbalance demonstrate field cancerization in histologically normal tissue adjacent to breast tumors. Int J Cancer 119, 108-116 (2006).
2A. Trujillo, K. A., et al. Breast Field Cancerization: Isolation and Comparison of Telomerase Expressing Cells in Tumor and Tumor Adjacent, Histologically Normal Breast Tissue. Mol Cancer Res (2011).
3A. Hines, W. C., Fajardo, A. M., Joste, N. E., Bisoffi, M. & Griffith, J. K. Quantitative and spatial measurements of telomerase reverse transcriptase expression within normal and malignant human breast tissues. Mol Cancer Res 3, 503-509 (2005).
4A. Trujillo, K. A., et al. Markers of fibrosis and epithelial to mesenchymal transition demonstrate field cancerization in histologically normal tissue adjacent to breast tumors. Int J Cancer (2010).

1. Substantially purified exosomes which induce apoptosis in breast cancer cells and which are derived from histologically normal breast tissue cells that are obtained from tumor-adjacent normal breast tissue.

2. The substantially purified exosomes of claim 1, wherein the exosomes are derived from a cultured medium of histologically normal breast tissue cells obtained from tumor-adjacent normal breast tissue located approximately 3 to about 10 cm from a breast cancer tumor.

3. The substantially purified exosomes of claim 1, wherein the histologically normal breast tissue cells are obtained from normal breast tissue located approximately 5 cm from a breast cancer tumor.

4. The substantially purified exosomes of claim 1, wherein the histologically normal breast tissue cells are obtained from breast branching epithelium (terminal duct lobular units (TDLUs)) and/or surrounding stroma.

5. The substantially purified exosomes of claim 1, wherein the histologically normal breast tissue cells are selected from the group consisting of luminal epithelial cells, myoepithelial cells, fibroblasts, immune cells, endothelial cells and extracellular matrix cells.

6. The substantially purified exosomes of claim 1, wherein the histologically normal breast tissue cells are fibroblasts.

7. The substantially purified exosomes of claim 1, wherein the breast cancer tumor is associated with invasive ductal carcinoma, ductal carcinoma in situ (DCIS) or invasive lobular carcinoma.

8. The substantially purified exosomes of claim 1, wherein the breast cancer tumor expresses or is associated with one or more breast tumor-associated antigens or compositions selected from the group consisting of epidermal growth factor receptor EGFR, HER/neu, CR1, M18, M39, HER2 antigen (p185HER2), polymorphic epithelial mucin (PEM) (Hilkens et al, 1992, Trends in Bio. Chem. Sci. 17:359), carcinoma embryonic antigen (CEA), prostate specific antigen (PSA) Erb B2 antigen, gross cystic disease fluid protein-15 (GCDFP-15), lactose dehydrogenase (LDH), circulating tumor DNA CA 15-3, carcinoembryonic antigen (CEA), cancer antigen 125 (CA 125), Survivin, MUC1, CD44, CD24, oestrogen receptor alpha (ERα), CA15-3, TPA, TPS, Urokinase plasminogen activator (uPA) and plasminogen activator inhibitor (PAI-1).

9. The substantially purified exosomes of claim 1, wherein the exosomes are loaded with a small molecule, antisense oligonucleotide, siRNA, peptide, protein or antibody that inhibits the growth of, or induces apoptosis in, breast cancer cells.

10. (canceled)

11. (canceled)

12. (canceled)

13. (canceled)

14. (canceled)

15. (canceled)

16. (canceled)

17. (canceled)

18. (canceled)

19. A pharmaceutical formulation which is useful in the treatment of breast cancer and which comprises a therapeutically effective amount of the substantially purified exosomes of claim 1 and, optionally, an additional anti-cancer agent and a pharmaceutically acceptable excipient.

20. (canceled)

21. The pharmaceutical formulation of claim 19, wherein the formulation comprises one or more additional anticancer agents selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, platinum coordination complexes including everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, NO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, and mixtures thereof.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A method for treating a neoplastic cancer in an animal, comprising: administering to the animal in need thereof an effective quantity of exosomes produced by a fibroblast cell line obtained from histologically normal tissue within a neoplastic cancer affected organ of said animal.

36. The method of claim 35 wherein the step of administering comprises direct application to, or direct injection into a tumor.

37. The method of claim 35 wherein the step of administering comprises intravenous administration or delivery via the lymphatic circulation system.

38. The method of claim 35 wherein the animal is a human.

39. The method of claim 35 wherein the animal is a non-human.

40. The method of claim 35 wherein the fibroblast cell line is derived from tissue at a distance of greater than about 2 cm from a tumor margin in the cancer affected organ in the animal.

41. The method of claim 35 wherein the fibroblast cell line is derived from tissue at a distance of between about 3-6 cm from a tumor in the cancer affected organ in the animal.

42. The method of claim 35 wherein the cancer affected organ in the animal is a mammary organ.

43. The method of claim 35 wherein the histologically normal tissue of the cancer affected organ is located outside of the field cancerized tissue.

44.-73. (canceled)
